(19) United States
(12) Patent Application Publication
(10) Pub. No.: US 2022/0040294 A1
(43) Pub. Date: Feb. 10, 2022

(54) ADMINISTRATION OF VACCINES TO SITES OF TEMPORALLY INDUCED CELL-MEDIATED HYPERSENSITIVITY REACTIONS, TO FACILITATE THE DEVELOPMENT OF PROTECTIVE SENSITIZATION AGAINST INFECTIOUS DISEASES AND CANCERS

(71) Applicants: Robert E. COIFMAN, Millville, NJ (US); Catherine F. YANG, Elk Grove, CA (US)

(72) Inventors: Robert E. COIFMAN, Millville, NJ (US); Catherine F. YANG, Elk Grove, CA (US)

(21) Appl. No.: 17/394,506

(22) Filed: Aug. 5, 2021

Related U.S. Application Data
(60) Provisional application No. 63/063,072, filed on Aug. 7, 2020.

(51) Int. Cl. A61K 39/35

(52) U.S. Cl. CPC A61K 39/35; A61K 2039/545

(57) ABSTRACT

Administration of an agent or substance to the site of a subject of a temporally interacting cell-mediated allergic reaction is used to enhance the resulting level of protective sensitization.

### RELATED APPLICATION

This application claims the benefit of U.S. Provisional Application 63/063,072, filed on Aug. 7, 2020, the contents of which are hereby incorporated herein by reference in its entirety

### FIELD

The present disclosure relates to the deliberate induction of a cell-mediated hypersensitivity reaction, clinically known as allergic contact dermatitis, to modify the biology of the skin to which a vaccine is applied, to bias the response to that vaccine toward stronger protective immunity.

### BACKGROUND

A vaccine is defined by the CDC as “A product that stimulates a person's immune system to produce immunity to a specific disease, protecting the person from that disease.” Vaccines act by either reinforcing an existing state of immune system responsiveness or triggering immunomodulation from one state of responsiveness to another. Vaccines to protect patients from allergic diseases are designed to induce immunomodulation from pathological states of sensitization to immunological tolerance. Vaccines to protect against infectious diseases can either induce immunomodulation from immunological naivete to protective sensitization or boost or enhance an existing state of protective sensitization. Vaccines to protect against cancer are designed to induce immunomodulation from tolerance of a patient's own cancer cells to a state of protective immunity.

While most vaccines are currently given by mouth or subcutaneous or by intramuscular injection, the first effective vaccine against an infectious disease (Edward Jenner's vaccine against smallpox), was given by multiple punctures through a drop of active vaccine into the epidermis and dermis. The skin is again becoming a target of interest for the administration of vaccines to protect against both infectious and neoplastic diseases to exploit the presence and organization of cells and cell types within the skin whose ability to facilitate protective sensitization is an evolutionary task of our skin to protect those of our tissues that live inside it.

Since the publication of 2012 M. B. M. Teunissen edited vol. 351 of Springer's Current Topics in Microbiology and Immunology on Intradermal Immunization (“Teunissen”), there have been quantitative advances in the field in the form of additional and improved applications of the principles and methods described in that volume but we know of nothing qualitatively new in the field.

### BRIEF SUMMARY OF THE INVENTION

A cutaneous cell-mediated allergic reaction creates a localized inflammatory dendritic cell and cytokine milieu, replicating many of the micro-environmental characteristics believed to facilitate protective sensitization as illustrated in FIG. 1. The present invention is directed to administering vaccines against infectious diseases and cancers in association with natural or induced cutaneous cell-mediated allergic reactions as a way to enhance the resulting protective sensitization.

### BRIEF DESCRIPTION OF THE FIGURE

FIG. 1 is a graphic representation from Teunissen et al. of the function of the adaptive immune system in the skin. In FIG. 1, dendritic cells (DC) present in normal skin under normal conditions are shown at upper left. Additional dendritic cell populations induced and/or recruited by an inflammatory reaction, in the case of the present invention by a cutaneous cell-mediated allergic reaction to a substance that need not be antigenically related. In the center of FIG. 1 is the tryst between a dendritic cell presenting antigen and a naive T-lymphocyte receiving it. At right are a number of pathways by which that T-lymphocyte could mature and function, depending on the type of dendritic cell and the micro-environmental chemical milieu in which this liaison takes place.

### DETAILED DESCRIPTION OF THE INVENTION

The teleological rationale for use of the skin and the nose as target tissues for vaccines designed to elicit protective sensitization is the following, in comparison to the alternatives of oro-gastric topical administration and parenteral (subcutaneous or intramuscular) injection. The main immunological task of the oro-gastro-intestinal system is to facilitate a combination of local and systemic tolerance to potentially allergenic foods so that our bodies can use them as sources of nutrition. This feature of this group of tissues is exploited in emerging forms of sublingual and oral immunotherapy to achieve immunomodulation from sensitization to tolerance in allergic disease states. The only successful oral vaccines for infectious disease are live vaccines that replicate in the host, for which the oral route is simply a convenient method of administration.

The two jobs of the parenteral immune system are immune surveillance against mutations that could potentially be cancerous AND the preservation of tolerance of self. Vaccines have been developed for injection beneath the skin or into muscle to switch the immune response in both directions. Switching of immunologic responsiveness from naivete to protective sensitization is exploited by injecting non-living vaccines to provoke protective sensitization against infectious diseases. The same route can be used to guide the immune response from sensitization to tolerance in allergic diseases. Protective sensitization is the primary job of the immune system in the skin, though very slow release of a peanut vaccine into the skin from a patch has been shown to shift the systemic immune response to greater tolerance. As the mechanisms by which the skin facilitates sensitization are identified, vaccine developers are working to develop ways to exploit them. If one thinks of the number of viral infections for which the nose is the portal of entry one will appreciate the evolutionary pressure on the lining membranes of the nasal cavity to develop dendritic cell trafficking patterns and cytokine patterns that favor sensitization. The propensity of this tissue to develop allergic sensitization is demonstrated by the frequency and severity of nasal allergy to pollens and other airborne particulates.

A delayed hypersensitivity reaction and a cell-mediated allergic reaction are different terms for the same thing and will be treated as synonymous in this document. Similarly, dendritic cells are migratory antigen-presenting cells and the two terms will also be treated in this document as synonymous.

It is contemplated that there may be therapeutic changes other than changes in dendritic cell population and cytokine milieu. As used herein, the term “temporal, or temporally interacting” is meant to include: Sufficiently related by time of administration that the induced cell-mediated reaction alters the immune response of the host compared to that observed or expected to the same vaccine in the absence of the induced cell-mediated reaction.

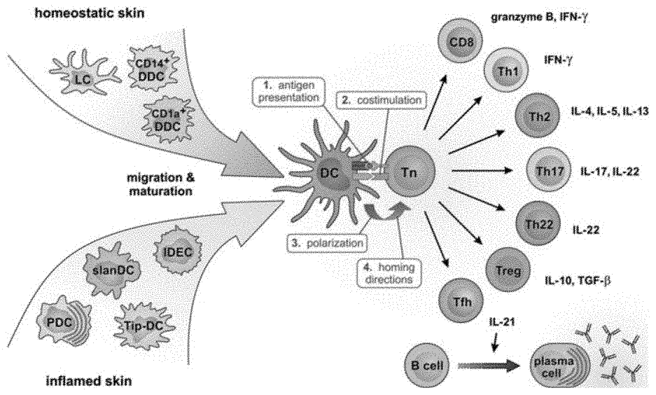

FIG. 1 is a graphic representation of the function of the adaptive immune system in the skin, reproduced from the above-cited book on intradermal immunization. At the left it shows dendritic cells of which different populations normally inhabit both the epidermis and the dermis. These recognize and take up molecules or particles of an antigen which they then present to naive T lymphocytes which then mature along one of what are shown here as seven different pathways.

Both the direction and the intensity of the lymphocyte response are determined by which population of dendritic cells picks up and delivers the antigen to the naive lymphocytes at the right middle of the figure, and the cytokine milieu in which they do it.

The dendritic cells shown at the upper left are those present in skin under normal or homeostatic conditions. Those at lower left are the populations generated or recruited in response to inflammation. The physiology of the primary function of the immune system in the skin is that if an infectious organism breaks through the physical barrier or the non-living outer layers of the skin, its presence is recognized by the innate immune system which elicits a local inflammatory reaction. This provokes the recruitment and generation of the new populations of dendritic cells shown at lower left and also alters the local cytokine milieu. The function of those new populations of dendritic cells and altered cytokine milieu is to facilitate a protective immune response to the infecting organism that will be recalled to defend the host if the provoking infection persists or any time that infecting organism is encountered again.

Immune responses to infection are typically complex, involving different types of antibodies and also cell-mediated immunity. All types of antibodies are not protective. Total antibody titers are easy to measure for large numbers of persons who have either recovered from an infectious disease or been immunized against it. The ease of measurement of levels of antibody with different functionalities depends on the ease of replication of the function to me measured. Measurement of cell-mediated immunity is difficult for geographically scattered patient populations as it requires either adequate numbers of healthy and viable cells from clinically relevant sources or intact individuals. When correlations can be established between antibody content or titer and protective cell-mediated immunity in small numbers of individuals, those antibody levels or titers are then tracked over populations as surrogate markers of protective cell mediated immunity.

The immune system identifies and attacks cancer cells by recognizing the same differences in surface chemistry by which it recognizes and attacks infecting microorganisms. In persons with normal immune systems most cells that develop pre-cancerous mutations never survive to cause disease. In immunologically normal individuals those abnormal cells that successfully reproduce to become cancers have done so by developing mechanisms to trick and/or bypass the normal protective function of the host immune system.

Interventions that have been studied to improve the ability of vaccines to induce protective sensitization to both infectious diseases and cancers include:

1. Use of the intradermal route, to take advantage of the evolutionary biasing of the immune system in the skin toward protective sensitization.
2. Pre-administration or co-administration of substances designed to elicit or mimic the inflammatory changes that accompany successful natural development of protective sensitization to agents causing local infection.
3. In U.S. patent application Ser. No. 16/664,849, by the present inventors, filed on Oct. 26, 2019, now U.S. Published Application 2020-0222531 A1, precipitation of particles of water-insoluble antigen within the dermis and in a size range efficiently taken up by naive dendritic cells by macropinocytosis, as a pharmaceutically acceptable water-miscible solvent in which they are administered is diluted by tissue water.

The present invention includes the induction of a localized, in most applications mild, natural cell-mediated allergic reaction and exploiting the immunomodulatory effect of that cell-mediated allergic reaction will have to bias the host response to a vaccine in the direction of protective sensitization compared to what it would be without the induced cell-mediated allergic reaction. Immune reactions are not all or none but span a wide range of intensity. The goal of this invention is to achieve clinically effective immunomodulation from naivete (in infections disease) or tolerance (in cancer) to a protective combination of cell-mediated and/or humoral immunity with a minimum of morbidity from the procedure making it desirable to induce the mildest exogenous cell mediated allergic reaction needed to predictably and reliably achieve the desired immunomodulation. There may be embodiments of the invention and there may be individual patients who fail to respond to what are determined to be generally effective doses of non-natural sensitizing agents, for example, who will respond if the intensity of the provoking cell-mediated allergic reaction is changed. Possible mechanisms of action of the invention include but are not limited to local generation and/or stimulation of the influx of sensitizing dendritic cell populations and the creation in response to the inducing cell-mediated reaction of a cytokine milieu more favorable to the development of protective sensitization. Future technologies might make it possible to elicit and monitor a localized delayed hypersensitivity reactions in internal tissues, but at this time visual evaluation is only practical on the skin and in the nose. Their natural exposure to the outside world of potential sources of infection has given both of these tissues an evolutionarily predisposition to the development of allergic sensitization, which the induced cell mediated allergic reaction of this invention is designed to enhance. In each case and for each tissue the purpose of the local induced delayed hypersensitivity reaction is to create the immunological micro-environmental changes depicted in FIG. 1 to favor and facilitate immunomodulation from naivete or tolerance to protective sensitization.

The present invention captures all uses in which the induced cell mediated reaction alters either or both of the dendritic cell populations and the cytokine milieu with (for the dendritic cells) or in (for cytokine milieu) which the immune system of the host responds to the vaccine.

The most familiar cell mediated allergic reactions of this type are allergic contact dermatitis to poison oak and poison ivy, which are highly cross-reactive with each other and able to sensitize ˜85% of Americans. According to example embodiments, it may be preferable to use non-natural sensitizers to which potential recipients would not have the broad range of prior level of sensitization resulting from variable natural exposure to poison oak and poison ivy. A historically used non-natural contact sensitizer was dinitrochlorobenzene (DNCB). It was used as a topical treatment for warts. It created a local cell-mediated allergic reaction in the micro-environment in which antigen-presenting host dendritic cells were exposed to virus-specific antigens at the margins of a benign, virus-induced tumor that does not have the skill set of many malignant tumors to evade local immune system recognition. The dendritic cells that have picked up tumor cell surface antigens present them to the naive T-lymphocytes illustrated in the center of FIG. 1 whether a DNCB-induced cell-mediated reaction is present or not, but in the absence of the immunomodulating cell mediated allergic reaction the wart survives and thrives. When the cell-mediated DNCB reaction is present, it biases the resulting immune response sufficiently in the direction of protective sensitization to generate a reaction that eliminates the tumor. The outcome is the same shifting of immune system response toward protective sensitization that our invention is designed to replicates for vaccines.

DNCB was withdrawn from clinical use because of its bacterial mutagenicity in the Ames test (Happle R The Potential Hazards of Dinitrochlorobenzene. Arch Dermatol. 1985; 121(3):330-332. doi:10.1001/archderm.1985.01660030052016). It is not clear whether DNCB would in fact carry a risk of carcinogenicity because the immunomodulatory effect of DNCB is to enhance the type of immune response most strongly associated with protection against cancers. Happle names a number of similarly immunostimulatory non-natural substances that do not fail the Ames test, however.

The induction of a natural cell-mediated immune reaction to a non-natural universal sensitizer to which the vast majority of the population has not been previously exposed would be the most practical way to develop a standardized protocol for the implementation of this invention. Everyone with genetically normal cell-mediated immunologic reactivity could be expected to respond, and in most cases, to respond to a standardized dose, even if their genetically normal cell-mediated immune system was immunosuppressed. The goal of these embodiments of the invention is to achieve the same boost of protective immunity to an exogenously administered vaccine that direct application of DNCB achieves for a common wart. For immunotherapy of cancer the exogenously administered vaccine could be made from the patient's own tumor antigens either with or without intermediate modification to increase their antigenicity.

A concurrent or temporally proximate induced cell-mediated reaction in a location distant from a site of vaccine administration could potentially induce micro-environmental changes at a remote vaccine administration site that could induce protective immunomodulation if it causes no detectable microscopic or chemical change in the microenvironment of the vaccine administration site. Applicant submits that it's possible that a concurrent or temporally proximate induced cell-mediated reaction could induce a systemic effect on vaccine response and that any such case of distant induction of protective sensitization without identifiable changes in injection site histology of chemistry would still fall within the scope of this invention.

There may be applications of the present invention for which a booster or recall cell mediated immune response is more effective than an initial sensitization response at creating a dendritic cell and cytokine milieu favorable to protective sensitization. It will be technically easiest to standardize a recall challenge dose for a non-natural sensitizer to which almost the only individuals previously exposed will be those who have previously received the treatment of this invention.

Elicitation of a recall reaction to a natural sensitizer such as poison ivy may be an effective way to apply the present invention to a cancer patient so severely immunosuppressed as to be unable to mount a response to a sensitizer to which they had not been previously exposed. The problem with general use of poison ivy is that the topical dose needed to induce the same degree of recall reaction was observed to vary by a factor of 39,000 (Coifman R E, Yang C F, Tolerance to poison ivy following vaccine delivery by precipitation, Annals of Allergy, Asthma and Immunology 2019(March); 122:331-33),

DNCB and other non-natural universal contact sensitizers cause a plurality of changes in dendritic cell populations and cytokine milieu. Changes will be different for sensitizing and recall reactions and also depend on the intensity of the induced cell-mediated reaction and the time in the course of that reaction at which the vaccine is administered. The different cellular and micro-environmental chemical effects of the different elements of the response to induced cell-mediated allergic reactions will have different time courses. When DNCB is repeatedly painted on a wart the patient benefits from a cumulative net positive balance of its net therapeutic and anti-therapeutic effects. Where the antigen to which one wants to induce protective sensitization is only administered at one or a small number of discrete points in time, however, the temporal relationship between administration of the sensitizing agent and the vaccine may be important. As different vaccines that work by different mechanisms may have different “sweet spots” in their dose and temporal relationships to dosing of the sensitizing agent, it may take systematic small scale trial and error guided by reaction site cytology to identify these “sweet spots” of dosage and timing.

In embodiments of the present invention, the substance inducing the cell mediated reaction may be applied to the intended vaccine administration site immediately prior to vaccine administration or a specified time interval prior to vaccine administration that may be determined to be most efficacious for a particular combination of sensitizer and vaccine. There may be applications for which the patient is sensitized sufficiently prior to vaccine administration for the dendritic cell and cytokine milieu of the intended site of vaccine administration to return to normal but that the sensitizer (to which by this time the patient is already sensitized) is applied to the intended vaccine administration site any of before, concurrent with, or even a specified time after administration of the vaccine. It is unlikely but theoretically possible for an initial dose of a sensitizer to which the recipient does not already have cell mediated immunity to achieve protective immunomodulation if it is not applied until AFTER the vaccine is administered, but any such instance will still fall within the scope if this invention.

This application is submitted in the era of COVID-19, when numerous candidate vaccines of numerous types are being fast-tracked to expedite the discovery of something that works. The inventors believe that many of these tracks can be bent in the direction of that goal by:

1) Exploring the effect of an induced local cell-mediated allergic reaction on the response to intracutaneously or intranasally administered COVID-19 vaccines (as well as other vaccines for infectious diseases and cancers) toward more vigorous protective sensitization.

2) Performing small scale comparative trials, beginning in animal models when available, of the dose and timing variables described herein. Therapeutic induced cell-mediated vaccine application site reactions could be initiated prior to, concurrent with, or possibly even subsequent to administration of the vaccine to which the desired outcome is protective sensitization.

3) Expect that the optimal relationship of dose and timing may be different for different types of vaccines and different particle sizes which may be taken up by dendritic cells by different mechanisms. (Xiang S D, Scholzen A, Minigo G, et al. Pathogen recognition and development of particulate vaccines: does size matter? Methods. 2006; 40:1e9.)

The present invention includes methods of biasing a response of a vaccine recipient's immune system toward protective sensitization by induction of an immunomodulating cell-mediated allergic reaction in a specified temporal relationship and a specified spatial relationship to administration of the vaccine to the recipient, wherein the specified temporal relationship includes induction of the immunomodulating cell-mediated allergic reaction from 0 to 21 days before vaccine administration to 0 to 21 days after vaccine administration. According to example embodiments, the specified temporal relationship includes induction of the immunomodulating cell-mediated allergic reaction from 0 to 24 hours before vaccine administration to 0 to 24 hours after vaccine administration.

The specified spatial relationship may require vaccine administration at the same site as the immunomodulating cell-mediated allergic reaction, at a proximate site close enough to reflect histological or microenvironmental chemical changes caused by the immunomodulating cell mediated allergic reaction, at a site with no identifiable histological or microenvironmental chemical changes but drained by the same regional lymph nodes, or independent of body location.

Provided herein by way of non-limiting example embodiments are methods that include the induction of cutaneous cell-mediated allergic reactions to modulate the response of the immune system to vaccines administered to areas of skin affected by said induced cell-mediated allergic reactions. At the cellular and micro-environmental chemical level these reactions consist of evolving series of inflammatory and reparative changes in both cell populations and cytokine milieu. Many steps of these processes replicate micro-environmental conditions known or believed to be associated with the enhancement of protective sensitization. The present invention relates to the use of a temporally interacting cutaneous cell-mediated allergic reaction to a substance that is not necessarily antigenically related to the vaccine being given.

A non-limiting embodiment of this invention is topical administration of a standardized sensitizing or recall dose of a non-natural universal sensitizer to the skin or nasal mucosa (standardized to maximize likelihood of efficacy with minimum risk of unnecessary severity) followed either immediately or at a prescribed subsequent interval by a topically penetrating, locally precipitating formulation of the vaccine.

In a non-limiting variation of the above embodiment the agent provoking an intranasal or cutaneous cell mediated allergic reaction can be applied topically but the vaccine applied by injection into the nasal mucosa, dermis or sub-dermal space.

According to non-limiting example embodiments, the agent to induce a cell-mediated allergic reaction is an antigen provoking a surface cell-mediated allergic reaction

According to non-limiting example embodiments, the subject may have been previously sensitized to the antigen provoking the surface cell-mediated allergic reaction. According to other non-limiting example embodiments, the subject has not been previously sensitized to the agent inducing the cell-mediated allergic reaction.

According to non-limiting example embodiments, the antigen inducing the enhancing cell-mediated allergic reaction is antigenically related to the immunizing antigenic material. In other embodiments, the antigen provoking the cell-mediated allergic reaction is not antigenically related to some component of the immunizing antigenic material.

According to non-limiting examples, the substance provoking the cutaneous cell-mediated allergic reaction is administered prior to administration of the vaccine whose protective sensitization that cutaneous cell-mediated allergic reaction is intended to enhance.

The agent provoking the cutaneous cell-mediated allergic reaction may be administered temporally with the vaccine whose protective sensitization the cutaneous cell-mediated allergic reaction is intended to enhance.

According to example embodiments, the agent is administered cutaneously and the agent inducing the cutaneous cell-mediated allergic reaction is administered prior to, subsequently or subsequent to administration of the vaccine whose protective sensitization the cutaneous cell-mediated allergic reaction is intended to enhance.

In example embodiments, the locally induced therapeutic immunomodulatory cell mediated reaction is internal.

Also provided are methods of enhancing protective immunity in a subject induced by a vaccine, that include inducing a local cell-mediated allergic reaction in the subject by administrating a sensitizing agent to the subject; and administering a vaccine to the subject. The local cell-mediated allergic reaction may be induced from 0 to 21 days before vaccine administration to 0 to 21 days after vaccine administration or from 0 to 24 hours before vaccine administration to 0 to 24 hours after vaccine administration.

Also provided are methods of enhancing protective immunity in a subject induced by a vaccine, that include administering to the subject an agent cutaneously or intra-nasally to a site of the subject to induce a cell-mediated allergic reaction, at the cellular and micro-environmental chemical level at the site. Example methods also include administering a vaccine to the subject, for which the subject's immune system processes in tissues affected by a cell-mediated allergic reaction, to the subject before during or after administration of the agent to induce a cell-mediated allergic reaction.

According to non-limiting example embodiments, a sensitizing agent may be applied topically to skin or lining membranes of nose of the subject. A sensitizing agent may be applied to the subject internally. According to example embodiments, the sensitizing agent is applied to the subject by injection.

In example embodiments, the vaccine may be applied to a same site on the patient as the agent provoking the cell-mediated allergic reaction. In example embodiments, the vaccine may be applied to a different site on the patient than the agent provoking the cell-mediated allergic reaction, or it may be applied to a similar location on the patient. The vaccine administration may be at a proximate site close enough to the site of the immunomodulating cell-mediated allergic reaction to reflect histological or microenvironmental chemical changes caused by the immunomodulating cell mediated allergic reaction or at a site with drained by the same regional lymph nodes.

The present invention is intended to encompass any application of the present invention in which the application sites of the agent provoking the reaction and the vaccine are or are not identical, whether the vaccine is injected beneath the site of application of a provoking agent to the skin or whether the two sites are far apart.

The present invention includes use of the cellular and micro-environmental chemical changes of a local cell-mediated allergic reaction in a subject to enhance the protective immunity induced by a locally administered vaccine temporally with said cell-mediated reaction. The sensitizing agent may be applied topically to skin or lining membranes of nose of the subject to induce the cell-mediated allergic reaction in the subject.

Also provided herein are methods of enhancing the protective immunity induced by a locally administered vaccine that includes administering an agent to induce a cell-mediated hypersensitivity reaction, at the cellular and micro-environmental chemical level, and administering a vaccine to a location in a human or other multi-cellular animal recipient at which a delayed hypersensitivity reaction either has been induced, is concurrently being induced or will shortly be induced. According to example embodiments, sensitizing agent may be applied topically to skin or lining membranes of nose of the subject.

In non-limiting examples of the present methods, the agent to induce a cell-mediated hypersensitivity reaction is an antigen provoking a cutaneous cell-mediated allergic reaction. In non-limiting examples, the subject may have been previously sensitized to the antigen provoking the cutaneous cell-mediated allergic reaction. In other examples, the subject has not been previously sensitized to the antigen provoking the cell-mediated allergic reaction.

According to further non-limiting examples, the antigen inducing the enhancing cell-mediated allergic reaction may be antigenically related to the immunizing antigenic material. In other examples, the antigen provoking the cell-mediated allergic reaction is not antigenically related to some component of the immunizing antigenic material.

In non-limiting example embodiments, the substance provoking the cutaneous cell-mediated allergic reaction is administered prior to administration of the vaccine whose protective sensitization that cutaneous cell-mediated allergic reaction is intended to enhance. In other examples, the agent provoking the cutaneous cell-mediated allergic reaction is administered temporally, (i.e., concurrently or one immediately after the other) as the vaccine whose protective sensitization the cutaneous cell-mediated allergic reaction is intended to enhance. In further examples, the agent provoking the cutaneous cell-mediated allergic reaction is administered either prior to or subsequent to administration of the vaccine whose protective sensitization that cutaneous cell-mediated allergic reaction is intended to enhance.

It is conceivable that changes in immune system responsiveness caused by an induced cell-mediated allergic reaction could have the same beneficial immunomodulatory effect for vaccines administered other than intracutaneously, topically to the nasal mucosa or topically to the skin in a vehicle such as dimethyl sulfoxide capable of transporting it into the skin, a non-limiting example being subcutaneous injection beneath skin in which s cell-mediated allergic reaction has been or is concurrently being induced.

Other non-limiting embodiments of the invention may allow induction of the immunomodulating cell-mediated allergic reaction anywhere in the area of the body draining to the same regional lymph nodes or at any body location chosen to minimize the resulting inconvenience to the recipient, which would be the case for a systemic immunomodulatory effect.

Although the present disclosure has been described in example embodiments, additional modifications and variations would be apparent to those skilled in the art. It is therefore to be understood that the present disclosure herein may be practiced other than as specifically described, Thus, the present embodiments should be considered in all respects as illustrative, and not restrictive. Accordingly, it is intended that such changes and modifications fall within the scope of the present disclosure as defined by the claims appended hereto.

We claim:

1. A method of biasing a response of a vaccine recipient's immune system toward protective sensitization by induction of an immunomodulating cell-mediated allergic reaction in a specified temporal relationship and a specified spatial relationship to administration of the vaccine to the recipient,
wherein the specified temporal relationship includes induction of the immunomodulating cell-mediated allergic reaction from 0 to 21 days before vaccine administration to 0 to 21 days after vaccine administration, and
wherein the specified spatial relationship requires vaccine administration at a proximate site close enough to the site of the immunomodulating cell-mediated allergic reaction to reflect histological or microenvironmental chemical changes caused by the immunomodulating cell mediated allergic reaction or at a site with drained by the same regional lymph nodes.

2. The method of claim 1, wherein the temporal relationship includes induction of the immunomodulating cell-mediated allergic reaction from 0 to 24 hours before vaccine administration to 0 to 24 hours after vaccine administration.

3. The method of claim 1, wherein the specified spatial relationship requires vaccine administration at the same site of the immunomodulating cell-mediated allergic reaction.

4. A method of enhancing protective immunity in a subject induced by a vaccine comprising
administering to the subject an agent cutaneously or intra-nasally to a site of the subject to induce a cell-mediated allergic reaction, at the cellular and micro-environmental chemical level at the site, and
administering a vaccine to the subject, for which the subject's immune system processes in tissues affected by a cell-mediated allergic reaction, to the subject before during or after administration of the agent to induce a cell-mediated allergic reaction.

5. The method of claim 4, wherein the agent to induce a cell-mediated allergic reaction is an antigen provoking a surface cell-mediated allergic reaction.

6. The method of claim 5, wherein the subject has been previously sensitized to the antigen provoking the surface cell-mediated allergic reaction.

7. The method of claim 5, wherein the subject has not been previously sensitized to the antigen provoking the cell-mediated allergic reaction.

8. The method of claim 5, wherein the antigen provoking the cell-mediated allergic reaction is antigenically related to the immunizing antigenic material.

9. The method of claim 5, wherein the antigen provoking the cell-mediated allergic reaction is not antigenically related to the immunizing antigenic material.

10. The method of claim 4, wherein the agent is administered cutaneously, and the agent inducing the cutaneous cell-mediated allergic reaction is administered prior to administration of the vaccine whose protective sensitization the cutaneous cell-mediated allergic reaction is intended to enhance.

11. The method of claim 4, wherein the agent is administered cutaneously, and the agent inducing the cutaneous cell-mediated allergic reaction is administered temporally with administration of the vaccine whose protective sensitization the cutaneous cell-mediated allergic reaction is intended to enhance.

12. The method of claim 4, wherein the agent is administered cutaneously, and the agent inducing the cutaneous cell-mediated allergic reaction is administered subsequent to administration of the vaccine whose protective sensitization the cutaneous cell-mediated allergic reaction is intended to enhance.

13. The method of claim 4, wherein the induced cell mediated reaction is internal.

14. A method of enhancing protective immunity in a subject induced by a locally administered vaccine, comprising
inducing a local cell-mediated allergic reaction in the subject by administrating a sensitizing agent to the subject; and
administering a vaccine to the subject, wherein said local cell-mediated allergic reaction is induced 0 to 24 hours before vaccine administration to 0 to 24 hours after vaccine administration.

15. The method of claim 14, wherein the sensitizing agent is applied topically to skin or lining membranes of nose of the subject.

16. The method of claim 14, wherein the sensitizing agent is applied to the subject internally.

17. The method of claim 14, wherein the sensitizing agent is applied to the subject by injection.

18. The method of claim 14, wherein the vaccine is applied to a same site on the subject as the agent provoking the cell-mediated allergic reaction.

19. The method of claim 14, wherein the vaccine is applied to a different site on the subject than the agent provoking the cell-mediated allergic reaction.
